(19) United States
(12) Patent Application Publication
(10) Pub. No.: US 2010/0144727 A1
(43) Pub. Date: Jun. 10, 2010

(54) Oxazolidine and Morpholine Carboxamide Derivatives as P2X7 Modulators

(76) Inventors: Paul John Beswick, Essex (GB); Daryl Simon Walter, Essex (GB)

Correspondence Address: GlaxoSmithKline;GLOBAL PATENTS -US, UW2220, P. O. BOX 1539, KING OF PRUSSIA, PA, 19406-0939, US

(21) Appl. No.: 12/593,357

(22) PCT Filed: Mar. 20, 2008
(86) PCT No.: PCT/EP08/53431

§ 371 Date: Sep. 28, 2009

(30) Foreign Application Priority Data
Mar. 29, 2007 (GB) 0706206.0
Mar. 18, 2008 (GB) 0805048.6

(51) Int. Cl. A61K 31/421; C07D 263/20; C07D 265/32; A61K 31/5375; A61P 25/00; A61P 29/00

(52) U.S. Cl. 514/230.8; 548/230; 544/168; 514/376

(57) ABSTRACT

The present invention relates to a compound of formula (I):

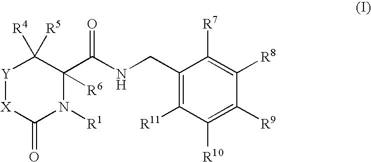

The compounds modulate P2X7 receptor function and are capable of antagonizing the effects of ATP at the P2X7 receptor. The invention also provides the use of such compounds, or pharmaceutical compositions thereof, in the treatment or prevention of disorders mediated by the P2X7 receptor, for example pain, inflammation or a neurodegenerative disease, in particular pain such as inflammatory pain, neuropathic pain or visceral pain.

The present invention relates to heterocyclic amide derivatives which modulate P2X7 receptor function and are capable of antagonizing the effects of ATP at the P2X7 receptor (“P2X7 receptor antagonists”); to processes for their preparation; to pharmaceutical compositions containing them; and to the use of such compounds in therapy.

The P2X7 receptor is a ligand-gated ion-channel which is expressed in cells of the hematopoietic lineage, e.g. macrophages, microglia, mast cells, and lymphocytes (T and B) (see, for example, Collo, et al. Neuropharmacology, Vol. 36, pp 1277-1283 (1997)), and is activated by extracellular nucleotides, particularly adenosine triphosphate (ATP). Activation of P2X7 receptors has been implicated in giant cell formation, degranulation, cytolytic cell death, CD62L shedding, regulation of cell proliferation, and release of proinflammatory cytokines such as interleukin 1 (IL-1β) and tumour necrosis factor (TNFα) (e.g. Hide, et al. Journal of Neurochemistry, Vol 75., pp 965-972 (2000)). P2X7 receptors are also located on antigen presenting cells, keratinocytes, parotid cells, hepatocytes, erythrocytes, erythroleukaemic cells, monocytes, fibroblasts, bone marrow cells, neurones, and renal mesangial cells. Furthermore, the P2X7 receptor is expressed by presynaptic terminals in the central and peripheral nervous systems and has been shown to mediate glutamate release in glial cells (Anderson, C. et al. Drug. Dev. Res., Vol. 50, page 92 (2000)).

The localisation of the P2X7 receptor to key cells of the immune system, coupled with its ability to release important inflammatory mediators from these cells suggests a potential role of P2X7 receptor antagonists in the treatment of a wide range of diseases including pain and neurodegenerative disorders. Recent preclinical in vivo studies have directly implicated the P2X7 receptor in both inflammatory and neuropathic pain (Dell'Antonio et al., Neurosci. Lett., 327, pp 87-90, 2002, Chessell, I P., et al., Pain, 114, pp 386-396, 2005) while there is in vitro evidence that P2X7 receptors mediate microglial cell induced death of cortical neurons (Skaper, S. D., et al., Program No. 937.7. 2005 Abstract Viewer/Itinerary Planner. Washington, D.C.: Society for Neuroscience, 2005. Online). In addition, up-regulation of the P2X7 receptor has been observed around β-amyloid plaques in a mouse model of Alzheimer's disease (Parvathenani, L. et al. J. Biol. Chem., Vol. 278(15), pp 13309-13317, 2003).

Preparations 31 and 32 of WO 02/00631 A2 (Fujisawa Pharmaceutical Co., Ltd) disclose the preparation and synthetic use of N-benzyl ((3S)-4-benzyl-5-oxomorpholin-3-yl)amide. Reference Example 21 of EP 0 472 826 A2 (Kanebo Ltd) discloses the preparation and synthetic use of (S)-N-benzyl-5-oxo-3-morpholinecarboxamide.

The present invention provides compounds which modulate P2X7 receptor function and are capable of antagonizing the effects of ATP at the P2X7 receptor (“P2X7 receptor antagonists”).

A first aspect of the present invention provides a compound of formula (I) or a pharmaceutically acceptable salt thereof:

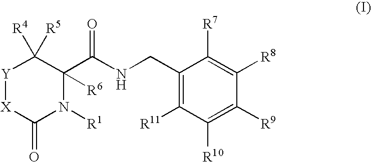

wherein:

R¹ represents C₁₋₆ alkyl, C₂₋₆ alkenyl, C₂₋₆ alkynyl, C₃₋₆ cycloalkyl, C₃₋₆ cycloalkylmethyl-, pyridinylmethyl- or benzyl, any of which is optionally substituted with 1, 2 or 3 halogen atoms; or an unsubstituted phenyl;

X represents O or —(CR²R³)—;

Y represents a bond or O;

such that when X is O, Y represents a bond; and when X is —(CR²R³)—, Y represents O;

R² and R³ independently represent hydrogen, C₁₋₆ alkyl, C₆₋₁₀ arylmethyl- or C₃₋₆ cycloalkylmethyl-; and any of said C₁₋₆ alkyl, C₆₋₁₀ arylmethyl- or C₃₋₆ cycloalkylmethyl- is optionally substituted with 1, 2 or 3 halogen (e.g. fluorine) atoms;

R⁴, R⁵ and R⁶ independently represent hydrogen, fluorine or methyl; and

R⁷, R⁸, R⁹, R¹⁰ and R¹¹ independently represent hydrogen, halogen (e.g. fluorine or chlorine), cyano, C₁₋₆ alkyl, C₂₋₆ alkenyl, C₂₋₆ alkynyl, C₃₋₆ cycloalkyl or phenyl, and any of said C₁₋₆ alkyl, C₂₋₆ alkenyl, C₂₋₆ alkynyl, C₃₋₆ cycloalkyl or phenyl is optionally substituted with 1, 2 or 3 halogen (e.g. fluorine) atoms; or R¹⁰ and R¹¹ together with the carbon atoms to which they are attached form a benzene ring which is optionally substituted with 1, 2 or 3 halogen (e.g. fluorine or chlorine) atoms;

with the proviso that when R⁷ and R¹¹ are both selected from hydrogen or fluorine, at least one of R⁸, R⁹ and R¹⁰ is a halogen atom.

A second aspect of the present invention provides a compound of formula (IA) or a pharmaceutically acceptable salt thereof, for use in therapy and/or for use in human or veterinary medicine:

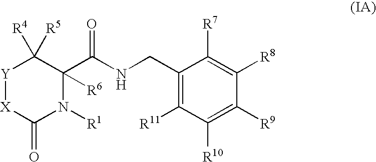

wherein:

R¹ represents C₁₋₆ alkyl, C₂₋₆ alkenyl, C₂₋₆ alkynyl, C₃₋₆ cycloalkyl, C₃₋₆ cycloalkylmethyl-, pyridinylmethyl- or benzyl, any of which is optionally substituted with 1, 2 or 3 halogen atoms; or an unsubstituted phenyl;

X represents O or —(CR²R³)—;

Y represents a bond or O;

such that when X is O, Y represents a bond; and when X is —(CR²R³)—, Y represents O;

R² and R³ independently represent hydrogen, C₁₋₆ alkyl, C₆₋₁₀ arylmethyl- or C₃₋₆ cycloalkylmethyl-; and any of said C₁₋₆ alkyl, C₆₋₁₀ arylmethyl- or C₃₋₆ cycloalkylmethyl- is optionally substituted with 1, 2 or 3 halogen (e.g. fluorine) atoms;

R⁴, R⁵ and R⁶ independently represent hydrogen, fluorine or methyl; and

R⁷, R⁸, R⁹, R¹⁰ and R¹¹ independently represent hydrogen, halogen (e.g. fluorine or chlorine), cyano, C₁₋₆ alkyl, C₂₋₆ alkenyl, C₂₋₆ alkynyl, C₃₋₆ cycloalkyl or phenyl, and any of said C₁₋₆ alkyl, C₂₋₆ alkenyl, C₂₋₆ alkynyl, C₃₋₆ cycloalkyl or phenyl is optionally substituted with 1, 2 or 3 halogen (e.g. fluorine) atoms; or R¹⁰ and R¹¹ together with the carbon atoms to which they are attached form a benzene ring which is optionally substituted with 1, 2 or 3 halogen (e.g. fluorine or chlorine) atoms;

with the proviso that when R⁷ and R¹¹ are both selected from hydrogen or fluorine, at least one of R⁸, R⁹ and R¹⁰ is a halogen atom, or not more than one of R⁸, R⁹ and R¹⁰ is a CF₃ group.

In a particular embodiment, in a compound of formula (IA) or a salt thereof, when R⁷ and R¹¹ are both selected from hydrogen or fluorine, at least one of R⁸, R⁹ and R¹⁰ is a halogen atom.

All embodiments, e.g. particular or preferable features or aspects, of the invention (e.g. embodiments of the compound or salt of the invention and/or of pharmaceutical compositions and/or uses thereof) which are disclosed herein in relation to a compound of formula (I) or a salt thereof, are also hereby disclosed and contemplated in relation to a compound of formula (IA) or a salt thereof, to the extent appropriate or possible, with all necessary changes having been made to the wording.

As used herein, the term “alkyl” (when used as a group or as part of a group) refers to a straight or branched hydrocarbon chain containing the specified number of carbon atoms. For example, C₁₋₆ alkyl means a straight or branched hydrocarbon chain containing at least 1 and at most 6 carbon atoms. Examples of alkyl include, but are not limited to; methyl (Me), ethyl (Et), n-propyl, i-propyl, n-hexyl and i-hexyl.

As used herein, the term “alkenyl” refers to a straight or branched hydrocarbon chain containing the specified number of carbon atoms wherein at least one carbon-carbon bond is a double bond. Examples of alkenyl include, but are not limited to ethenyl, propenyl, n-butenyl, i-butenyl, n-pentenyl and i-pentenyl.

As used herein, the term “alkynyl” refers to a straight or branched hydrocarbon chain containing the specified number of carbon atoms wherein at least one carbon-carbon bond is a triple bond. Examples of alkynyl include, but are not limited to ethynyl, propynyl, butynyl, i-pentynyl, n-pentynyl, i-hexynyl and n-hexynyl.

The term ‘cycloalkyl’ unless otherwise stated means a closed 3 to 6 membered non-aromatic ring, for example cyclopropyl, cyclobutyl, cyclopentyl or cyclohexyl.

The term ‘aryl’ as used herein refers to a C₆₋₁₀ monocyclic or bicyclic hydrocarbon ring wherein at least one ring is aromatic. Examples of such groups include phenyl and naphthyl.

The term ‘halogen’ is used herein to describe, unless otherwise stated, a group selected from fluorine, chlorine, bromine or iodine.

It is to be understood that the present invention covers and discloses all possible combinations of particular, preferred, suitable, or other embodiments of groups (e.g. of X, Y, R¹, R², R³, R⁴, R⁵, R⁶, R⁷, R⁸, R⁹, R¹⁰ and/or R¹¹), e.g. all possible combinations of embodiments of different groups, which embodiments are described herein.

In certain particular embodiments of the invention, R¹ represents unsubstituted C₁₋₆ alkyl (e.g. methyl, ethyl, n-propyl or i-propyl), C₂₋₆ alkenyl, C₂₋₆ alkynyl, C₃₋₆ cycloalkyl or benzyl.

In a particular embodiment, R¹ represents unsubstituted methyl, ethyl or benzyl.

Preferably, R¹ represents unsubstituted methyl or ethyl (i.e. methyl or ethyl).

In certain particular embodiments of the invention, R² and R³ independently represent hydrogen or unsubstituted C₁₋₆ alkyl, benzyl or C₃₋₆ cycloalkylmethyl-.

Preferably, R² and R³ both represent hydrogen.

In one embodiment of the invention, X represents O and Y represents a bond. In an alternative embodiment of the invention, X represents —(CR²R³)— and Y represents O; in which case, preferably X represents —CH₂— and Y represents O.

In a particular embodiment of the invention, R⁴ and R⁵ can in particular both represent hydrogen. In a particular embodiment, R⁶ represents hydrogen.

Preferably, R⁴, R⁵ and R⁶ all represent hydrogen.

In a particular embodiment of the invention, R⁷, R⁸, R⁹, R¹⁰ and R¹¹ independently represent hydrogen, halogen (e.g. fluorine or chlorine), cyano, trifluoromethyl or unsubstituted C₁₋₆ alkyl; or R¹⁰ and R¹¹ together with the carbon atoms to which they are attached form an unsubstituted benzene ring. In a more particular embodiment, R⁷, R⁸, R⁹, R¹⁰ and R¹¹ independently represent hydrogen, halogen (e.g. fluorine or chlorine), cyano, methyl or trifluoromethyl; or R¹⁰ and R¹¹ together with the carbon atoms to which they are attached form an unsubstituted benzene ring. In a still more particular embodiment, R⁷, R⁸, R⁹, R¹⁹ and R¹¹ independently represent hydrogen, halogen (e.g. fluorine or chlorine), methyl or trifluoromethyl; or R¹⁰ and R¹¹ together with the carbon atoms to which they are attached form an unsubstituted benzene ring.

In a yet more particular embodiment, R⁷, R⁸, R⁹, R¹⁰ and R¹¹ independently represent hydrogen, chlorine, fluorine, bromine, methyl or trifluoromethyl; in particular hydrogen, chlorine, fluorine, methyl or trifluoromethyl.

In all embodiments of the invention herein described, with respect to a compound of formula (IA) or a salt thereof, when R⁷ and R¹¹ are both selected from hydrogen or fluorine, at least one of R⁸, R⁹ and R¹⁰ is a halogen atom, or not more than one of R⁸, R⁹ and R¹⁰ is a CF₃ group.

In all embodiments of the invention herein described, with respect to a compound of formula (I) or a salt thereof, when R⁷ and R¹¹ are both selected from hydrogen or fluorine, at least one of R⁸, R⁹ and R¹⁰ is a halogen atom.

In a particular embodiment of the invention herein described, with respect to a compound of formula (I) or (IA) or a salt thereof, when R⁷ and R¹¹ are both selected from hydrogen or fluorine, at least one of R⁸, R⁹ and R¹⁰ is a halogen atom, and not more than one of R⁸, R⁹ and R¹⁰ is a CF₃ group.

In a particular embodiment, R⁷ is hydrogen, R¹¹ is fluorine or chlorine, and R⁸, R⁹ and R¹⁰ independently represent hydrogen, chlorine, fluorine or trifluoromethyl. In a more particular embodiment, R⁷ is hydrogen, R¹¹ is fluorine or chlorine, one or two (e.g. two) of R⁸, R⁹ and R¹⁰ are hydrogen, and one or two (e.g. one) of R⁸, R⁹ and R¹⁰ independently represent chlorine, fluorine or trifluoromethyl. In a still more particular embodiment:

R⁷, R⁸ and R⁹ are hydrogen, R¹⁰ is trifluoromethyl, and R¹¹ is chlorine, or

R⁷, R⁸ and R¹⁰ are hydrogen, and R⁹ and R¹¹ are chlorine, or

R⁷, R⁸ and R¹⁰ are hydrogen, R⁹ is fluorine, and R¹¹ is chlorine, or

R⁷ and R⁸ are hydrogen, and R⁹, R¹⁰ and R¹¹ are fluorine.

In a particular embodiment, R⁷ is hydrogen, R¹¹ is chlorine, and R⁸, R⁹ and R¹⁰ independently represent hydrogen, chlorine, fluorine or trifluoromethyl. In a more particular embodiment, R⁷ is hydrogen, R¹¹ is chlorine, one or two (e.g. two) of R⁸, R⁹ and R¹⁰ are hydrogen, and one or two (e.g. one) of R⁸, R⁹ and R¹⁰ independently represent chlorine, fluorine or trifluoromethyl. In a preferred embodiment:

R⁷, R⁸ and R⁹ are hydrogen, R¹⁰ is trifluoromethyl, and R¹¹ is chlorine, or

R⁷, R⁸ and R¹⁰ are hydrogen, and R⁹ and R¹¹ are chlorine, or

R⁷, R⁸ and R¹⁰ are hydrogen, R⁹ is fluorine, and R¹¹ is chlorine.

Preferably, R⁷, R⁸ and R⁹ are hydrogen, R¹⁰ is trifluoromethyl, and R¹¹ is chlorine, or R⁷, R⁸ and R¹⁰ are hydrogen, and R⁹ and R¹¹ are chlorine.

More preferably, R⁷, R⁸ and R⁹ are hydrogen, R¹⁰ is trifluoromethyl, and R¹¹ is chlorine.

In one particular embodiment of the invention, there is provided a compound of formula (I) or (IA), or a pharmaceutically acceptable salt thereof, wherein:

R¹ represents unsubstituted methyl, ethyl or benzyl (in particular R¹ can represent (unsubstituted) methyl or ethyl);

X represents O or —CH₂—;

Y represents a bond or O;

such that when X is O, Y represents a bond; and when X is —CH₂—, Y represents O;

R⁴, R⁵ and R⁶ all represent hydrogen; and

R⁷, R⁸, R⁹, R¹⁰ and R¹¹ independently represent hydrogen, chlorine, fluorine, bromine, methyl or trifluoromethyl; or R¹⁰ and R¹¹ together with the carbon atoms to which they are attached form an unsubstituted benzene ring;

with the proviso that when R⁷ and R¹¹ are both selected from hydrogen or fluorine, at least one of R⁸, R⁹ and R¹⁰ is a halogen atom, or not more than one of R⁸, R⁹ and R¹⁰ is a CF₃ group (in formula (I), or in a particular embodiment of formula (IA), when R⁷ and R¹¹ are both selected from hydrogen or fluorine, at least one of R⁸, R⁹ and R¹⁰ is a halogen atom).

A particular aspect of the invention provides a compound selected from examples E1 to E10, as shown below and/or as described by name below.

A preferred aspect of the invention provides:
(4S)-N-[(2-chloro-4-fluorophenyl)methyl]-3-ethyl-2-oxo-1,3-oxazolidine-4-carboxamide

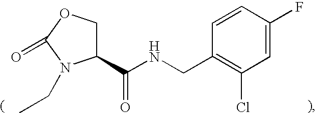

for example having an enantiomeric excess of greater than 70% (e.g. see Example 1), or
N-{[2-chloro-3-(trifluoromethyl)phenyl]methyl}-3-methyl-2-oxo-1,3-oxazolidine-4-carboxamide

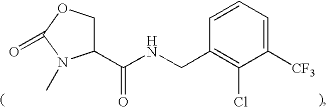

or
N-{[2-chloro-3-(trifluoromethyl)phenyl]methyl}-4-methyl-5-oxo-3-morpholinecarboxamide

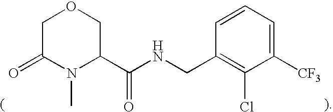

In a particular embodiment, the N-{[2-chloro-3-(trifluoromethyl)phenyl]methyl}-3-methyl-2-oxo-1,3-oxazolidine-4-carboxamide

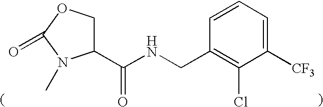

is: the (4R)-isomer, such as the (4R)-isomer having an enantiomeric excess of greater than 70% (e.g. see Example 3); or a racemate; or preferably the (4S)-isomer

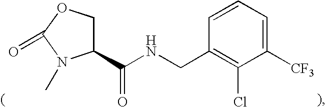

preferably the (4S)-isomer having an enantiomeric excess of greater than 70% (e.g. see Example 4).

In a preferred embodiment, the N-{[2-chloro-3-(trifluoromethyl)phenyl]methyl}-4-methyl-5-oxo-3-morpholinecarboxamide

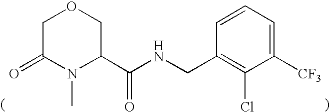

is in a form obtainable or prepared from N-{[(1,1-dimethylethyl)oxy]carbonyl}-N-methyl-L-serine (e.g. see Example 10).

A further particular aspect of the present invention provides a compound of formula (IB) or a pharmaceutically acceptable salt thereof:

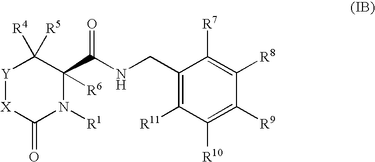

wherein:

R¹ represents C₁₋₄ alkyl or C₃₋₄ cycloalkyl, any of which is optionally substituted with 1, 2 or 3 halogen (e.g. fluorine) atoms,

and X, Y, R², R³, R⁴, R⁵, R⁶, R⁷, R⁸, R⁹, R¹⁰ and R¹¹ are as defined herein for the compound of formula (I) or salt thereof or for the compound of formula (IA) or salt thereof,

and wherein more than 50% (e.g. more than 70%, in particular more than 90%, such as more than 95%) by molarity of the compound of formula (IB) or the pharmaceutically acceptable salt thereof has the indicated stereochemistry at the ring-carbon atom bonded to R⁶.

In a particular embodiment, the compound of formula (IB) or the pharmaceutically acceptable salt thereof has an enantiomeric excess of greater than 70% (e.g. more than 80%, in particular more than 90%) with respect to the indicated stereochemistry at the ring-carbon atom bonded to R⁶.

In a particular embodiment, in a compound of formula (IB) or a salt thereof, R¹ represents unsubstituted C₁₋₄ alkyl or C₃₋₄ cycloalkyl; more particularly (unsubstituted) methyl, ethyl, n-propyl, i-propyl, cyclopropyl, or cyclobutyl; still more particularly (unsubstituted) methyl, ethyl, n-propyl or i-propyl.

In a most particular embodiment, in a compound of formula (IB) or a salt thereof, R¹ represents (unsubstituted) methyl or ethyl.

In a particular embodiment, in a compound of formula (IB) or a salt thereof, R², R³, R⁴, R⁵ and R⁶ all represent hydrogen.

All embodiments, e.g. particular or preferable features or aspects, of the invention (e.g. embodiments of the compound or salt of the invention and/or of pharmaceutical compositions and/or uses thereof) which are disclosed herein in relation to a compound of formula (I) or a salt thereof, are also hereby disclosed and contemplated in relation to a compound of formula (IB) or a salt thereof, to the extent appropriate or possible, with all necessary changes having been made to the wording.

An alternative particular aspect of the invention provides a compound of formula (I) or a pharmaceutically acceptable salt thereof, as disclosed herein, wherein the compound or salt is substantially racemic (e.g. racemic) at the ring-carbon atom bonded to R⁶.

Antagonists of P2X7 may be useful in preventing, treating, or ameliorating a variety of pain states (e.g. neuropathic pain, chronic inflammatory pain, and visceral pain), inflammation and neurodegeneration, in particular Alzheimer's disease. P2X7 antagonists may also constitute useful therapeutic agents in the management of rheumatoid arthritis and inflammatory bowel disease.

Compounds or salts of the present invention which modulate P2X7 receptor function and are capable of antagonizing the effects of ATP at the P2X7 receptor (“P2X7 receptor antagonists”) may be competitive antagonists, inverse agonists, or negative allosteric modulators of P2X7 receptor function.

Certain compounds of formula (I) may in some circumstances form acid addition salts thereof. It will be appreciated that for use in medicine compounds of formula (I) may be used as salts, in which case the salts should be pharmaceutically acceptable. Pharmaceutically acceptable salts include those described by Berge, Bighley and Monkhouse, J. Pharm. Sci., 1977, 66, 1-19. When a compound of the present invention is basic, in one embodiment a pharmaceutically acceptable salt is prepared from a pharmaceutically acceptable acid, such as an inorganic or organic acid, e.g. by admixture of the compound and the acid. Such acids include acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid, and the like. In a particular embodiment, the pharmaceutically acceptable acid is benzenesulfonic, camphorsulfonic, ethanesulfonic, hydrobromic, hydrochloric, methanesulfonic, nitric, phosphoric, sulfuric, or p-toluenesulfonic acid.

Examples of pharmaceutically acceptable salts include salts formed from maleic, fumaric, benzoic, ascorbic, pamoic, succinic, hydrochloric, sulfuric, bismethylenesalicylic, methanesulfonic, ethanedisulfonic, propionic, tartaric, salicylic, citric, gluconic, aspartic, stearic, palmitic, itaconic, glycolic, p-aminobenzoic, glutamic, benzenesulfonic, cyclohexylsulfamic, phosphoric and nitric acids.

The compounds of formula (I) or salts thereof may be prepared in crystalline or non-crystalline form, and, if crystalline, may optionally be solvated, e.g. as the hydrate. This invention includes within its scope stoichiometric solvates (e.g. hydrates) as well as compounds containing variable amounts of solvent (e.g. water).

Compounds of formula (I) or salts thereof are capable of existing in stereoisomeric forms (e.g. diastereomers and enantiomers) and the invention extends to each of these stereoisomeric forms and to mixtures thereof including racemates. The different stereoisomeric forms may be separated one from the other by the usual methods, or any given isomer may be obtained by stereospecific or asymmetric synthesis. In examples where the stereochemical composition of the final product has been determined by chiral HPLC (more specifically by methods (A), (B), (C) or (D) as set out in the Examples), the corresponding stereospecific name and structure have generally been assigned to the final product where the enantiomeric excess (e.e.) of said product is greater than 70%. Assignment of absolute stereochemistry is based on the known chirality of the starting material. In examples where the composition of the final product has not been characterised by chiral HPLC, the stereochemistry of the final product has not been indicated. However, the chirality of the main component of the product mixture of the compound or salt will generally be expected to reflect that of the starting material; and/or the enantiomeric excess will generally depend on the synthetic method used and is likely to be similar to that measured for an analogous example (where such an example exists). Thus compounds or salts shown in one chiral form are expected to be able to be prepared in the alternative chiral form using the appropriate starting material. Alternatively, if racemic starting materials are used, it would be expected that a racemic product would be produced and the single enantiomers could be separated by the usual methods. The invention also extends to any tautomeric forms and mixtures thereof.

The subject invention also includes isotopically-labeled compounds, which are identical to those recited in formula (I), or salts thereof, but for the fact that one or more atoms are replaced by an atom having an atomic mass or mass number different from the atomic mass or mass number most commonly found in nature. Examples of isotopes that can be incorporated into compounds or salts of the invention include isotopes of hydrogen, carbon, nitrogen, oxygen, phosphorous, fluorine, iodine, and chlorine, such as 3H, 11C, 14C, 18F, 123I and 125I.

Compounds of the present invention and pharmaceutically acceptable salts of said compounds that contain the aforementioned isotopes and/or other isotopes of other atoms are within the scope of the present invention. Isotopically-labeled compounds or salts of the present invention, for example those into which radioactive isotopes such as 3H, 14C are incorporated, are potentially useful in drug and/or substrate tissue distribution assays. Tritiated, i.e., 3H, and carbon-14, i.e., 14C, isotopes are for example optionally chosen for their ease of preparation and detectability. 11C and 8F isotopes are generally useful in PET (positron emission tomography), and 125I isotopes are generally useful in SPECT (single photon emission computerized tomography). PET and SPECT are useful in brain imaging. Further, substitution with heavier isotopes such as deuterium, i.e., 2H, can sometimes afford certain effects resulting from greater metabolic stability, for example increased in vivo half-life or reduced dosage requirements and, hence, may be chosen in some circumstances. Isotopically labeled compounds of formula (I) or salts thereof of this invention are in one embodiment and in some cases prepared by carrying out the procedures disclosed in the Schemes and/or in the Examples below, by substituting a readily available isotopically labeled reagent for a non-isotopically labeled reagent.

A further particular aspect of the invention provides a compound of formula (I) or a pharmaceutically acceptable salt thereof which is not a radioactive isotopically labeled compound or salt. In a particular embodiment, the compound or salt is not an isotopically labeled compound or salt.

### Preparation of Compounds

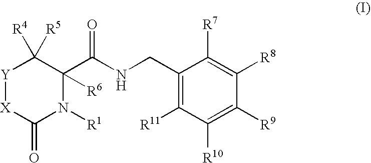

Compounds of formula (I), wherein the variables are as defined above, and salts and solvates thereof may be prepared by the methodology described hereinafter, constituting a further aspect of this invention.

According to a further aspect of the invention, there is provided a process for preparing a compound of formula (I) or a pharmaceutically acceptable salt thereof which comprises:

(a) Coupling of a carboxylic acid of formula (2) (or an activated derivative thereof) with an amine of formula (3) (see Scheme 1), wherein X, Y, R¹, R⁴, R⁵, R⁶, R⁷, R⁸, R⁹, R¹⁰, and R¹¹ are as defined above. Compounds (2) and (3) are optionally protected.

(b) The reaction of an amide of formula (4) with phosgene (or an equivalent reagent e.g. triphosgene) and a suitable base such as triethylamine in a suitable solvent such as dichloromethane and at a suitable temperature such as between 0° C. and 70° C. (see Scheme 2), wherein R¹, R⁴, R⁵, R⁶, R⁷, R⁸, R⁹, R¹⁰ and R¹¹ are as defined above. Compound (4), is optionally protected. Processes of this type have been described previously in the chemical literature.

(c) The reaction of an amide of formula (4) with a reagent of formula (5) and initial treatment with a suitable base such as triethylamine in a suitable solvent such as tetrahydrofuran and at a suitable temperature such as room temperature followed by treatment with another suitable base such as potassium hydroxide in a suitable solvent such as ethanol or isopropyl alcohol at a suitable temperature such as room temperature (see Scheme 3), wherein R¹, R², R³, R⁴, R⁵, R⁶, R⁷, R⁸, R⁹, R¹⁰ and R¹¹ are as defined above. L¹ and L² are suitable leaving groups such as halogen (e.g. bromine). Compound (4), is optionally protected. Processes of this type have been described previously in the chemical literature.

(d) Deprotecting a compound of formula (I) which is protected. Examples of protecting groups and the means for their removal can be found in T. W. Greene and P. G. M. Wuts ‘Protective Groups in Organic Synthesis’ (J. Wiley and Sons, 3^{rd} Ed. 1999).

(e) Interconversion of compounds of formula (I) to other compounds of formula (I). Examples of conventional interconversion procedures include epimerisation, oxidation, reduction, alkylation, aromatic substitution, nucleophilic substitution, amide coupling and ester hydrolysis.

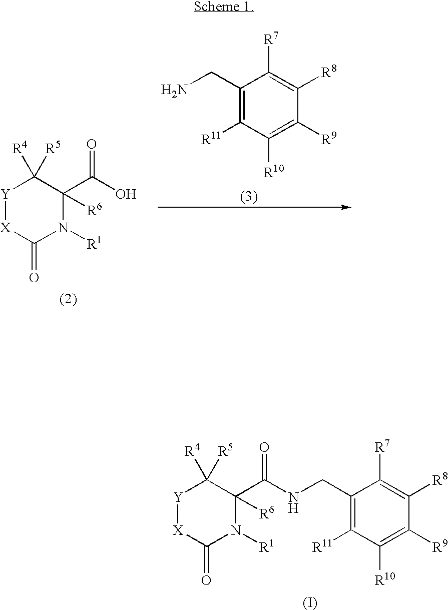

The coupling of an acid of formula (2) and an amine of formula (3) typically comprises the use of activating agents, such as N-(3-dimethylaminopropyl)-N′-ethylcarbodiimide hydrochloride or polymer-supported carbodiimide, 1-hydroxybenzotriazole (HOBT) or 1-Hydroxy-7-azabenzotriazole (HOAt), and optionally a suitable base such as a tertiary alkylamine (e.g. diisopropylethylamine, N-ethyl morpholine, triethylamine) or pyridine, in a suitable solvent such as DMF and/or dichloromethane and at a suitable temperature e.g. between 0° C. and room temperature. Alternatively the coupling of (2) and (3) may be accomplished by treatment with O-(7-Azabenzotriazol-1-yl)-N,N,N′,N′-tetramethyluronium hexafluorophosphate and a suitable tertiary alkylamine such as diisopropylethylamine in a suitable solvent such as dimethylformamide at a suitable temperature such as room temperature. Alternatively, the compound of formula (2) may be employed as an activated derivative (e.g. acid chloride, mixed anhydride, active ester (e.g. O-acyl-isourea)), and under such circumstances process (a) typically comprises treatment of said activated derivative with an amine (Ogliaruso, M. A.; Wolfe, J. F. in The Chemistry of Functional Groups (Ed. Patai, S.) Suppl. B: The Chemistry of Acid Derivatives, Pt. 1 (John Wiley and Sons, 1979), pp 442-8; Beckwith, A. L. J. in The Chemistry of Functional Groups (Ed. Patai, S.) Suppl. B: The Chemistry of Amides (Ed. Zabricky, J.) (John Wiley and Sons, 1970), pp 73 ff).

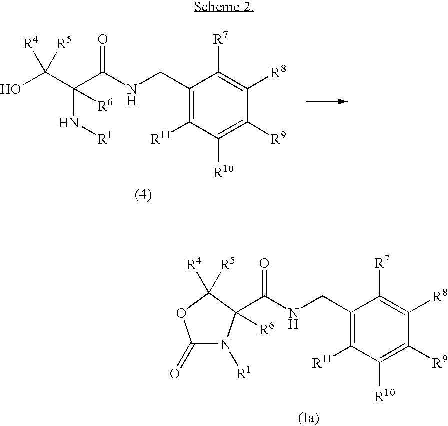

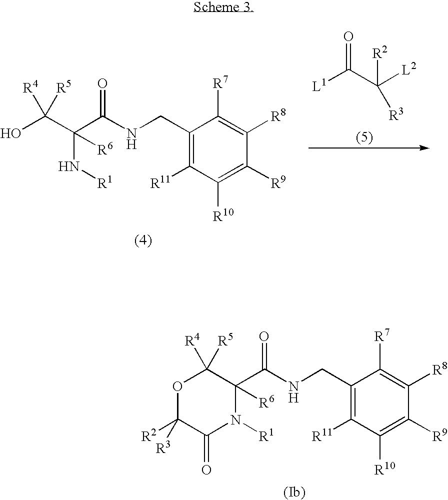

Representative methods for the preparation of compounds of formulas (2) and (4) are shown in Schemes 4-6 below:

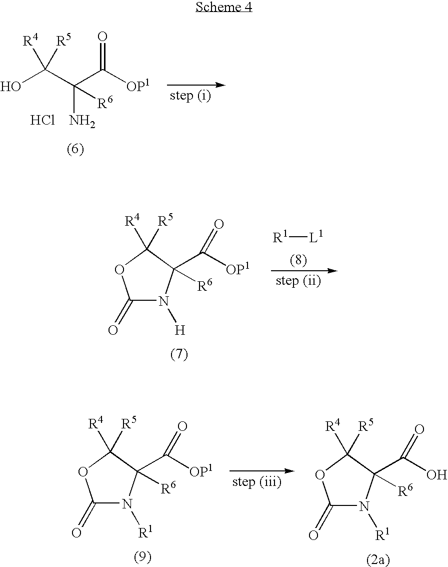

Wherein R¹, R⁴, R⁵, and R⁶ are as defined above. P¹ represents a suitable protecting group such as C₁₋₆ alkyl and L¹ represents a suitable leaving group such as a halogen (e.g. bromine or iodine).

Step (i) typically comprises treatment of (6) with phosgene or a suitable equivalent (e.g. triphosgene) in a suitable solvent such as tetrahydrofuran and at a suitable temperature such as between room temperature and 70° C.

Step (ii) typically comprises treatment of (7) with a base such as sodium hydride and an alkylating agent (8) such as an alkyl halide in a suitable solvent such as dimethylformamide at a suitable temperature such as between 0° C. and room temperature.

Deprotection step (iii) typically comprises a standard procedure for conversion of a carboxylic ester to an acid, such as use of an appropriate hydroxide salt (e.g. sodium hydroxide) in an appropriate solvent such as methanol at a suitable temperature such as 0° C.

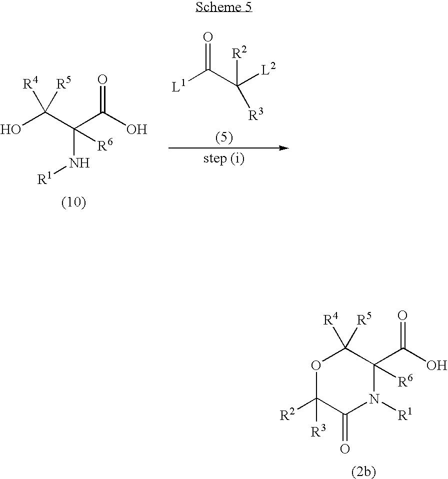

Wherein R¹, R², R³, R⁴, R⁵, and R⁶ are as defined above. L¹ and L² represent suitable leaving groups such as a halogen (e.g. chlorine, bromine or iodine).

Step (i) typically comprises treatment of (10) with a suitable base such as potassium carbonate and (5) in a suitable solvent such as a mixture of water and tetrahydrofuran and at a suitable temperature such as between −5° C. and room temperature.

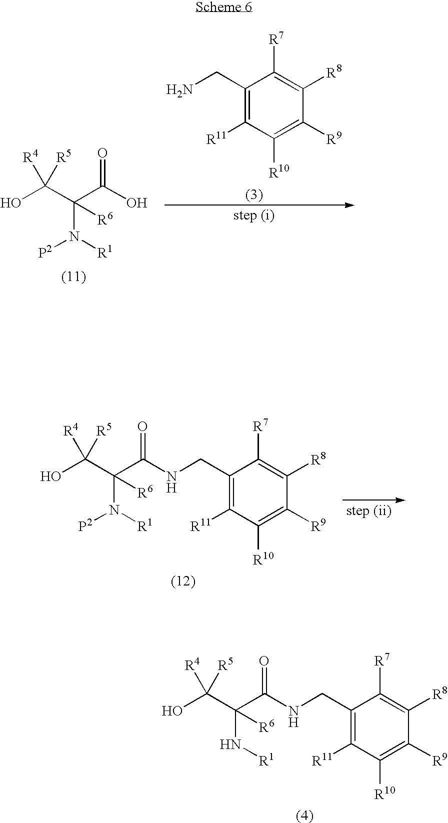

wherein R¹, R⁴, R⁵, R⁶, R⁷, R⁸, R⁹, R¹⁰, and R¹¹ are as defined above. P² represents a suitable amine protecting group such as C₁₋₄ alkoxy carbonyl.

Step (i) typically comprises coupling of a carboxylic acid of formula (11) (or an activated derivative thereof) with an amine of formula (3) (see Scheme 1 above) using suitable activating agents, such as N-(3-dimethylaminopropyl)-N′-ethylcarbodiimide hydrochloride and 1-hydroxybenzotriazole (HOBT) or 2-ethoxy-1-ethoxycarbonyl-1,2-dihydroquinoline in a suitable solvent such as dichloromethane and at a suitable temperature such as room temperature.

Deprotection step (ii) typically comprises a standard procedure for conversion of a alkoxy carbonyl protected amine to the corresponding free amine, such as use of an appropriate acid (e.g. trifluoroacetic acid or 1M hydrogen chloride in diethyl ether) in an appropriate solvent such as dichloromethane at a suitable temperature such as room temperature.

Compounds of the general formulae (3), (5), (6), (8), (10), and (11) are typically either available from commercial sources or can be prepared by a person skilled in the art using methods described in the chemical literature (or using analogous methods).

Where relevant, pharmaceutically acceptable salts may for example be prepared conventionally by reaction with the appropriate acid or acid derivative.

### Clinical Indications

It is believed that, as compounds or pharmaceutically acceptable salts of the present invention modulate P2X7 receptor function and are capable of antagonizing the effects of ATP at the P2X7 receptor (P2X7 receptor antagonists), they may be useful in the treatment of pain, including acute pain, chronic pain, chronic articular pain, musculoskeletal pain, neuropathic pain, inflammatory pain, visceral pain, pain associated with cancer, pain associated with migraine, tension headache and cluster headaches, pain associated with functional bowel disorders, lower back and neck pain, pain associated with sprains and strains, sympathetically maintained pain; myositis, pain associated with influenza or other viral infections such as the common cold, pain associated with rheumatic fever, pain associated with myocardial ischemia, post operative pain, cancer chemotherapy, headache, toothache and dysmenorrhea.

Chronic articular pain conditions include rheumatoid arthritis, osteoarthritis, rheumatoid spondylitis, gouty arthritis and juvenile arthritis.

Pain associated with functional bowel disorders includes non-ulcer dyspepsia, non-cardiac chest pain and irritable bowel syndrome.

Neuropathic pain syndromes include: diabetic neuropathy, sciatica, non-specific lower back pain, trigeminal neuralgia, multiple sclerosis pain, fibromyalgia, HIV-related neuropathy, post-herpetic neuralgia, trigeminal neuralgia, and pain resulting from physical trauma, amputation, phantom limb syndrome, spinal surgery, cancer, toxins or chronic inflammatory conditions. In addition, neuropathic pain conditions include pain associated with normally non-painful sensations such as “pins and needles” (paraesthesias and dysesthesias), increased sensitivity to touch (hyperesthesia), painful sensation following innocuous stimulation (dynamic, static, thermal or cold allodynia), increased sensitivity to noxious stimuli (thermal, cold, mechanical hyperalgesia), continuing pain sensation after removal of the stimulation (hyperpathia) or an absence of or deficit in selective sensory pathways (hypoalgesia).

Other conditions which could potentially be treated by compounds or pharmaceutically acceptable salts of the present invention include fever, inflammation, immunological diseases, abnormal platelet function diseases (e.g. occlusive vascular diseases), impotence or erectile dysfunction; bone disease characterised by abnormal bone metabolism or resorbtion; hemodynamic side effects of non-steroidal anti-inflammatory drugs (NSAID's) and cyclooxygenase-2 (COX-2) inhibitors, cardiovascular diseases; neurodegenerative diseases and/or neurodegeneration, neurodegeneration following trauma, tinnitus, dependence on a dependence-inducing agent such as opiods (e.g. morphine), CNS depressants (e.g. ethanol), psychostimulants (e.g. cocaine) and nicotine; complications of Type I diabetes, kidney dysfunction, liver dysfunction (e.g. hepatitis, cirrhosis), gastrointestinal dysfunction (e.g. diarrhoea), colon cancer, overactive bladder and urge incontinence. Depression and alcoholism could potentially also be treated by compounds or pharmaceutically acceptable salts of the present invention.

Inflammatory conditions include skin conditions (e.g. sunburn, burns, eczema, dermatitis, allergic dermatitis, psoriasis), meningitis, ophthalmic diseases such as glaucoma, retinitis, retinopathies, uveitis and of acute injury to the eye tissue (e.g. conjunctivitis), inflammatory lung disorders (e.g. asthma, bronchitis, emphysema, allergic rhinitis, respiratory distress syndrome, pigeon fancier's disease, farmer's lung, chronic obstructive pulmonary disease (COPD), airways hyperresponsiveness); gastrointestinal tract disorders (e.g. aphthous ulcer, Crohn's disease, atopic gastritis, gastritis varialoforme, ulcerative colitis, coeliac disease, regional ileitis, irritable bowel syndrome, inflammatory bowel disease, gastrointestinal reflux disease); organ transplantation and other conditions with an inflammatory component such as vascular disease, migraine, periarteritis nodosa, thyroiditis, aplastic anaemia, Hodgkin's disease, sclerodoma, myaesthenia gravis, multiple sclerosis, sorcoidosis, nephrotic syndrome, Bechet's syndrome, gingivitis, myocardial ischemia, pyrexia, systemic lupus erythematosus, polymyositis, tendinitis, bursitis, and Sjogren's syndrome.

Immunological diseases include autoimmune diseases, immunological deficiency diseases or organ transplantation.

Bone diseases characterised by abnormal bone metabolism or resorbtion include osteoporosis (especially postmenopausal osteoporosis), hyper-calcemia, hyperparathyroidism, Paget's bone diseases, osteolysis, hypercalcemia of malignancy with or without bone metastases, rheumatoid arthritis, periodontitis, osteoarthritis, ostealgia, osteopenia, cancer cacchexia, calculosis, lithiasis (especially urolithiasis), solid carcinoma, gout and ankylosing spondylitis, tendinitis and bursitis.

Cardiovascular diseases include hypertension or myocardiac ischemia; atherosclerosis; functional or organic venous insufficiency; varicose therapy; haemorrhoids; and shock states associated with a marked drop in arterial pressure (e.g. septic shock).

Neurodegenerative diseases include dementia, particularly degenerative dementia (including senile dementia, dementia with Lewy bodies, Alzheimer's disease, Pick's disease, Huntingdon's chorea, Parkinson's disease and Creutzfeldt-Jakob disease, Amyotrophic Lateral Sclerosis (ALS) and motor neuron disease); vascular dementia (including multi-infarct dementia); as well as dementia associated with intracranial space occupying lesions; trauma; infections and related conditions (including HIV infection, meningitis and shingles); metabolism; toxins; anoxia and vitamin deficiency; and mild cognitive impairment associated with ageing, particularly Age Associated Memory Impairment.

The compounds of formula (I) or pharmaceutically acceptable salts thereof may also be useful for neuroprotection and in the treatment of neurodegeneration following trauma such as stroke, cardiac arrest, pulmonary bypass, traumatic brain injury, spinal cord injury or the like.

The compounds or pharmaceutically acceptable salts of the present invention may also be useful in the treatment of malignant cell growth and/or metastasis, and myoblastic leukaemia.

Complications of Type 1 diabetes include diabetic microangiopathy, diabetic retinopathy, diabetic nephropathy, macular degeneration, glaucoma, nephrotic syndrome, aplastic anaemia, uveitis, Kawasaki disease and sarcoidosis.

Kidney dysfunction includes nephritis, glomerulonephritis, particularly mesangial proliferative glomerulonephritis and nephritic syndrome.

It is to be understood that reference to treatment includes both treatment of established symptoms and prophylactic treatment, unless explicitly stated otherwise.

According to a further aspect of the invention, we therefore provide a compound of formula (I) or a pharmaceutically acceptable salt thereof for use in therapy and/or for use in human or veterinary medicine.

According to another aspect of the invention, we provide a compound of formula (I) or a pharmaceutically acceptable salt thereof for use in the treatment or prevention (e.g. treatment) of a condition which is mediated by P2X7 receptors, for example a condition or disease disclosed herein (in particular pain, inflammation or a neurodegenerative disease, more particularly pain such as inflammatory pain, neuropathic pain or visceral pain), e.g. in a mammal such as a human or rodent e.g. human or rat e.g. human.

According to a further aspect of the invention, we provide a method of treating a human or animal (e.g. rodent e.g. rat) subject, for example a human subject, suffering from a condition which is mediated by P2X7 receptors [for example a condition or disease disclosed herein (in particular pain, inflammation or a neurodegenerative disease, more particularly pain such as inflammatory pain, neuropathic pain or visceral pain)] which comprises administering to said subject an effective amount of a compound of formula (I) or a pharmaceutically acceptable salt thereof.

According to a further aspect of the invention we provide a method of treating a human or animal (e.g. rodent e.g. rat) subject, for example a human subject, suffering from pain, inflammation, an immunological disease, a bone disease or a neurodegenerative disease (in particular pain, inflammation or a neurodegenerative disease, more particularly pain such as inflammatory pain, neuropathic pain or visceral pain), which method comprises administering to said subject an effective amount of a compound of formula (I) or a pharmaceutically acceptable salt thereof.

According to a yet further aspect of the invention we provide a method of treating a human or animal (e.g. rodent e.g. rat) subject, for example a human subject, suffering from inflammatory pain, neuropathic pain or visceral pain which method comprises administering to said subject an effective amount of a compound of formula (I) or a pharmaceutically acceptable salt thereof.

According to a further aspect of the invention we provide a method of treating a subject, for example a human subject, suffering from Alzheimer's disease which method comprises administering to said subject an effective amount of a compound of formula (I) or a pharmaceutically acceptable salt thereof.

According to another aspect of the invention, we provide the use of a compound of formula (I) or a pharmaceutically acceptable salt thereof for the manufacture of a medicament for the treatment or prevention (e.g. treatment) of a condition which is mediated by the action of P2X7 receptors, for example a condition or disease disclosed herein (in particular pain, inflammation or a neurodegenerative disease, more particularly pain such as inflammatory pain, neuropathic pain or visceral pain), e.g. in a mammal such as a human or rodent e.g. human or rat e.g. human.

According to another aspect of the invention we provide the use of a compound of formula (I) or a pharmaceutically acceptable salt thereof for the manufacture of a medicament for the treatment or prevention (e.g. treatment) of pain, inflammation, an immunological disease, a bone disease or a neurodegenerative disease (in particular pain, inflammation or a neurodegenerative disease, more particularly pain such as inflammatory pain, neuropathic pain or visceral pain), e.g. in a mammal such as a human or rodent e.g. human or rat e.g. human.

According to another aspect of the invention we provide the use of a compound of formula (I) or a pharmaceutically acceptable salt thereof for the manufacture of a medicament for the treatment or prevention (e.g. treatment) of inflammatory pain, neuropathic pain or visceral pain, e.g. in a mammal such as a human or rodent e.g. human or rat e.g. human.

In one aspect of the invention we provide the use of a compound of formula (I) or a pharmaceutically acceptable salt thereof for the manufacture of a medicament for the treatment or prevention (e.g. treatment) of Alzheimer's disease, e.g. in a mammal such as a human or rodent e.g. human or rat e.g. human.

In order to use a compound of formula (I) or a pharmaceutically acceptable salt thereof for the treatment of humans and other mammals it is normally formulated in accordance with standard pharmaceutical practice as a pharmaceutical composition. Therefore in another aspect of the invention there is provided a pharmaceutical composition comprising a compound of formula (I), or a pharmaceutically acceptable salt thereof, adapted for use in human or veterinary medicine.

In order to use a compound of formula (I) or a pharmaceutically acceptable salt thereof in therapy, it will normally be formulated into a pharmaceutical composition in accordance with standard pharmaceutical practice.

The present invention also provides a pharmaceutical composition, which comprises a compound of formula (I) or a pharmaceutically acceptable salt thereof, and optionally a pharmaceutically acceptable carrier or excipient.

The pharmaceutical composition may be for use in a method of treatment or in a use or in a treatment or prevention, as described herein.

A pharmaceutical composition of the invention, which may be prepared by admixture, for example at ambient temperature and atmospheric pressure, is usually adapted for oral, parenteral or rectal administration. As such, the pharmaceutical composition may be in the form of tablets, capsules, oral liquid preparations, powders, granules, lozenges, reconstitutable powders, injectable or infusable solutions or suspensions or suppositories. Orally administrable compositions are generally preferred.

Tablets and capsules for oral administration may be in unit dose form, and may contain conventional excipients, such as binding agents, fillers, tabletting lubricants, disintegrants and/or acceptable wetting agents. The tablets may be coated, e.g. according to methods well known in normal pharmaceutical practice.

Oral liquid preparations may be in the form of, for example, aqueous or oily suspension, solutions, emulsions, syrups or elixirs, or may be in the form of a dry product for reconstitution with water or other suitable vehicle before use. Such liquid preparations may contain conventional additives such as suspending agents, emulsifying agents, non-aqueous vehicles (which may include edible oils), preservatives, and, if desired, conventional flavourings or colourants.

For parenteral administration, fluid unit dosage forms are for example prepared utilising a compound of the invention or pharmaceutically acceptable salt thereof and a sterile vehicle. In one particular embodiment, the compound or salt, depending on the vehicle and concentration used, is either suspended or dissolved in the vehicle. In preparing solutions, the compound or salt can e.g. be dissolved for injection and filter sterilised before filling into a suitable vial or ampoule and sealing. In one embodiment, adjuvant(s) such as a local anaesthetic, a preservative and/or buffering agent are dissolved in the vehicle. To enhance the stability, the composition can for example be frozen after filling into the vial and the water removed under vacuum. Parenteral suspensions are typically prepared in substantially the same manner, except that the compound or salt is typically suspended in the vehicle instead of being dissolved, and sterilization is not usually readily accomplished by filtration. The compound or salt can be sterilised e.g. by exposure to ethylene oxide before suspension in a sterile vehicle. In a particular embodiment, a surfactant or wetting agent is included in the composition to facilitate uniform distribution of the compound or salt of the invention.

In one embodiment, the composition contains from 0.1% to 99% by weight, in particular from 10 to 60% by weight, of the active material (the compound or pharmaceutically acceptable salt of the invention), e.g. depending on the method of administration.

The dose of the compound or pharmaceutically acceptable salt thereof used in the treatment or prevention (e.g. treatment) of the aforementioned disorders/diseases/conditions may vary in the usual way with the seriousness of the disorders, the weight of the sufferer, and/or other similar factors. However, as a general guide, in one embodiment a unit dose of 0.05 to 1000 mg, for example 0.05 to 200 mg, such as 20 to 40 mg, of the compound or pharmaceutically acceptable salt of the invention (measured as the compound), may be used in one embodiment. In one embodiment, such a unit dose is for administration once a day e.g. to a mammal such as a human; alternatively such a unit dose may be for administration more than once (e.g. twice) a day e.g. to a mammal such as a human. Such therapy may extend for a number of weeks or months.

### Combinations

Compounds of formula (I) or salts thereof may be used in combination with other therapeutic agents, for example medicaments which are or may be useful in the treatment of the above mentioned disorders.

Suitable examples of other such therapeutic agents may include a β2-agonist (also known as β2 adrenoceptor agonists; e.g. formoterol) and/or a corticosteroid (e.g. budesonide, fluticasone (e.g. as propionate or furoate esters), mometasone (e.g. as furoate), beclomethasone (e.g. as 17-propionate or 17,21-dipropionate esters), ciclesonide, triamcinolone (e.g. as acetonide), flunisolide, rofleponide and butixocort (e.g. as propionate ester), for the treatment of respiratory disorders (such as asthma and chronic obstructive pulmonary disease (COPD)) as described in WO 2007/008155 and WO 2007/008157.

A further therapeutic agent may include a 3-hydroxy-3-methylglutaryl coenzyme A (HMG CoA) reductase inhibitor (e.g. atorvastatin, fluvastatin, lovastatin, pravastatin, rosuvastatin, and simvastatin) for the treatment of cardiovascular disorders (such as atherosclerosis) as described in WO 2006/083214.

A further therapeutic agent may include a non-steroid anti-inflammatory drug (NSAID; e.g. ibuprofen, naproxen, aspirin, celecoxib, diclofenac, etodolac, fenoprofen, indomethacin, ketoprofen, ketoralac, oxaprozin, nabumetone, sulindac, tolmetin, rofecoxib, valdecoxib, lumaricoxib, meloxicam, etoricoxiband and parecoxib) for the treatment of an inflammatory disease or disorder (such as rheumatoid arthritis or osteoarthritis) as described in WO 2005/025571.

A further therapeutic agent may include a tumour necrosis factor α (TNFα) inhibitor (e.g. Etanercept or an anti-TNFα antibody such as Infliximab and Adalimumab) for the treatment of an inflammatory disease or disorder (such as rheumatoid arthritis or osteoarthritis) as described in WO 2004/105798.

A further therapeutic agent may include 2-hydroxy-5-[[4-[(2-pyridinylamino) sulfonyl]phenyl]azo]benzoic acid (sulfasalazine) for the treatment of an inflammatory disease or disorder (such as rheumatoid arthritis) as described in WO 2004/105797.

A further therapeutic agent may include N-[4-[[(2,4-diamino-6-pteridinyl)methyl]methylamino]benzoyl]-L-glutamic acid (methotrexate) for the treatment of an inflammatory disease or disorder (such as rheumatoid arthritis) as described in WO 2004/105796.

A further therapeutic agent may include an inhibitor of pro TNFα convertase enzyme (TACE) for the treatment of an inflammatory disease or disorder (such as rheumatoid arthritis) as described in WO 2004/073704.

A further therapeutic agent may include:

a) sulfasalazine;

b) a statin, such as atorvastatin, lovastatin, pravastatin, simvastatin, fluvastatin, cerivastatin, crilvastatin, dalvastatin, rosuvastatin, tenivastatin, fluindostatin, velostatin, dalvastatin, nisvastatin, bervastatin, pitavastatin, rivastatin, glenvastatin, eptastatin, tenivastatin, flurastatin, rosuvastatin or itavastatin;

c) a glucocorticoid agent, such as dexamethasone, methylprednisolone, prednisolone, prednisone and hydrocortisone;

d) an inhibitor of p38 kinase;

e) an anti-IL-6-receptor antibody;

f) anakinra;

g) an anti-IL-1 monoclonal antibody;

h) an inhibitor of JAK3 protein tyrosine kinase;

i) an anti-macrophage colony stimulation factor (M-CSF) monoclonal antibody; or

j) an anti-CD20 monoclonal antibody, such as rituximab, PRO70769, HuMax-CD20 (Genmab AJS), AME-133 (Applied Molecular Evolution), or hA20 (Immunomedics, Inc.)

for the treatment of an IL-1 mediated disease (such as rheumatoid arthritis) as described in WO 2006/003517.

When the compounds are used in combination with other therapeutic agents, the compounds may be administered either sequentially or simultaneously by any convenient route.

The invention thus provides, in a further aspect, a combination comprising a compound of formula (I) or a pharmaceutically acceptable salt thereof together with a further therapeutic agent or agents, e.g. as described herein.

The combinations referred to above may conveniently be presented for use in the form of a pharmaceutical formulation and thus pharmaceutical formulations comprising a combination as defined above together with a pharmaceutically acceptable carrier or excipient comprise a further aspect of the invention. The individual components of such combinations may be administered either sequentially or simultaneously in separate or combined pharmaceutical formulations.

When a compound of formula (I) or a pharmaceutically acceptable salt thereof is used in combination with a second therapeutic agent active against the same disease state the dose of each compound may differ from that when the compound is used alone.

The following Descriptions and Examples illustrate the preparation of compounds of the invention but are not intended to be limiting.

### EXAMPLES

The general methods (a)-(e), along with the synthetic methods outlined in Schemes 1-6 above, for the preparation of compounds of the present invention are further illustrated by the following examples.

### Example 1

### N-[(2-chloro-4-fluorophenyl)methyl]-3-ethyl-2-oxo-1,3-oxazolidine-4-carboxamide (E1)

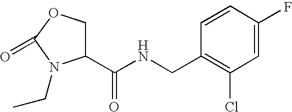

[(2-Chloro-4-fluorophenyl)methyl]amine (0.307 ml, 2.4 mmol), N-(3-dimethylaminopropyl)-N′-ethylcarbodiimide hydrochloride (0.458 g, 2.4 mmol), 1-hydroxybenzotriazole (0.323 g, 2.4 mmol), and N-ethyl morpholine (1.18 ml, 9.18 mmol), were added to crude 3-ethyl-2-oxo-1,3-oxazolidine-4-carboxylic acid (0.292 g, ˜1.84 mmol, prepared as described below) in dichloromethane (25 ml). The mixture was stirred at room temperature overnight under an atmosphere of argon. The mixture was then diluted with more dichloromethane (25 ml) and washed with saturated aqueous sodium hydrogen carbonate (50 ml). The organic layer was filtered through a hydrophobic frit and then evaporated to dryness giving a yellow solid/gum. The crude material was triturated with diethyl ether then dried to give a yellow solid (0.291 g). This material was then further purified by flash silica-gel column chromatography (eluting with 200 ml of a 95:5 mixture of dichloromethane and methanol respectively) to give N-[(2-chloro-4-fluorophenyl)methyl]-3-ethyl-2-oxo-1,3-oxazolidine-4-carboxamide (0.167 g) as a white solid after drying. LC/MS [M+H]⁺=301, retention time=2.20 minutes.

Enantiomeric excess=70.1%, as determined by chiral chromatography method D, indicative of (4S)-N-[(2-chloro-4-fluorophenyl)methyl]-3-ethyl-2-oxo-1,3-oxazolidine-4-carboxamide

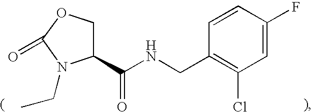

retention time=10.01 minutes

The 3-Ethyl-2-oxo-1,3-oxazolidine-4-carboxylic acid used in the above procedure was prepared as follows:

(i) A solution of triphosgene (1.9 g, 6.43 mmol) in tetrahydrofuran (6 ml) was added to a suspension of L-serine methyl ester hydrochloride (1 g, 6.43 mmol) in tetrahydrofuran (12 ml) at room temperature under argon. The mixture was heated at reflux for 5.5 hrs (all solids dissolved) and then cooled and concentrated to give a yellow oil (˜1.7 g). The crude material was purified by flash silica-gel column chromatography, eluting with 0-75% ethyl acetate in petroleum ether 40-60, to give methyl 2-oxo-1,3-oxazolidine-4-carboxylate as a colourless oil (0.77 g).

(ii) Methyl 2-oxo-1,3-oxazolidine-4-carboxylate (0.77 g, 5.31 mmol) was dissolved in dimethylformamide (5 ml) and added to a suspension of sodium hydride (60% in oil, 0.255 g, 6.37 mmol) in dimethylformamide (5 ml) at 0° C. under argon. The mixture was stirred at 0° C. for 0.5 hrs and then a further 5 ml of dimethylformamide was added to aid stirring. Ethyl iodide (0.509 ml, 6.37 mmol) was then added and the mixture was allowed to warm to room temperature and then stirred overnight. The solvent was evaporated and the resulting residue was purified by flash silica-gel column chromatography, eluting with 0-40% ethyl acetate in petroleum ether 40-60, to give methyl 3-ethyl-2-oxo-1,3-oxazolidine-4-carboxylate (0.250 g) as a pale yellow oil. This material was used in the next step without further purification.

(iii) 2N Aqueous sodium hydroxide (1 ml) was added dropwise to a solution of methyl 3-ethyl-2-oxo-1,3-oxazolidine-4-carboxylate (0.246 g, 1.42 mmol) in methanol (2 ml) at 0° C. The mixture was stirred at 0° C. for 4 hrs and then the methanol was evaporated in vacuo. The resulting aqueous layer was acidified using 2N aqueous hydrogen chloride (10 ml) and then this was extracted with dichloromethane (10 ml). The organic layer was separated using a hydrophobic frit and then discarded. The aqueous layer was evaporated to give crude 3-ethyl-2-oxo-1,3-oxazolidine-4-carboxylic acid which was used without further purification.

LC/MS [M+H]⁺=160, retention time=0.82 minutes.

### Example 2

### N-[(2-chloro-4-fluorophenyl)methyl]-3-ethyl-2-oxo-1,3-oxazolidine-4-carboxamide (E2)

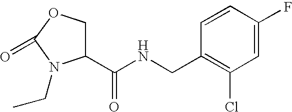

N-[(2-chloro-4-fluorophenyl)methyl]-3-ethyl-2-oxo-1,3-oxazolidine-4-carboxamide (E2) was prepared in a manner analogous to that described above in example 1 but using D-serine methyl ester hydrochloride in the place of L-serine methyl ester hydrochloride. LC/MS [M+H]⁺=301, retention time=2.20 minutes. Enantiomeric excess=68.7%, as determined by chiral chromatography method D, indicative of (4R)-N-[(2-chloro-4-fluorophenyl)methyl]-3-ethyl-2-oxo-1,3-oxazolidine-4-carboxamide

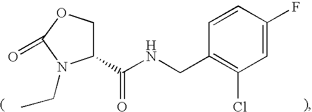

retention time=12.63 minutes

### Example 3

### N-{[2-chloro-3-(trifluoromethyl)phenyl]methyl}-3-methyl-2-oxo-1,3-oxazolidine-4-carboxamide (E3)

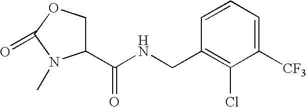

A suspension of 3-methyl-2-oxo-1,3-oxazolidine-4-carboxylic acid (0.200 g, ˜1 mmol, prepared as described below) in dichloromethane (5 ml) was treated with N-(3-dimethylaminopropyl)-N′-ethylcarbodiimide hydrochloride (0.230 g, 1.2 mmol), 1-hydroxybenzotriazole (0.162 g, 1.2 mmol), and N-ethyl morpholine (0.5 ml, 3 mmol) and then stirred at room temperature for 10 minutes. The mixture was then treated with a solution of {[2-chloro-3-(trifluoromethyl)phenyl]methyl}amine (0.210 g, 1 mmol) in dichloromethane (2 ml) and stirred for a further 1 hr at room temperature under an atmosphere of argon. The mixture was diluted with more dichloromethane and then washed sequentially with saturated aqueous sodium hydrogen carbonate solution, water, and brine. The organic layer was separated, dried, and evaporated to dryness giving crude material which was subsequently purified by flash silica-gel column chromatography (eluting with a 1:1 mixture of ethyl acetate and hexane) to give N-{[2-chloro-3-(trifluoromethyl)phenyl]methyl}-3-methyl-2-oxo-1,3-oxazolidine-4-carboxamide (0.068 g) as a colourless solid after drying. LC/MS [M+H]⁺=337/339, retention time=2.36 minutes.

Enantiomeric excess=90.9%, as determined by chiral chromatography method E, indicative of (4R)-N-{[2-chloro-3-(trifluoromethyl)phenyl]methyl}-3-methyl-2-oxo-1,3-oxazolidine-4-carboxamide, retention time=8.24 minutes

The 3-methyl-2-oxo-1,3-oxazolidine-4-carboxylic acid used in the above procedure was prepared in an analogous method to that used to prepare 3-ethyl-2-oxo-1,3-oxazolidine-4-carboxylic acid in example 1, but using D-serine methyl ester hydrochloride in the place of L-serine methyl ester hydrochloride and using methyl iodide in the place of ethyl iodide.

### Example 4

### N-{[2-chloro-3-(trifluoromethyl)phenyl]methyl}-3-methyl-2-oxo-1,3-oxazolidine-4-carboxamide (E4)

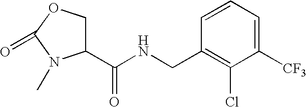

A suspension of crude N¹-{[2-chloro-3-(trifluoromethyl)phenyl]methyl}-N²-methylserinamide (0.620 g, 2 mmol, prepared as described below) in dichloromethane (30 ml) was stirred at 5° C. (ice-bath) under argon and treated with triethylamine (˜0.9 ml, 6 mmol). Stirring at 5° C. was continued for 5 minutes and then a solution of triphosgene (0.593 g, 2 mmol) in dichloromethane (5 ml) was added dropwise. Stirring at 5° C. was continued for 1 hr and then the solvent was evaporated to give the crude product. The above procedure was repeated on the same scale and then the combined crude products were purified by flash silica-gel column chromatography, eluting with a 1:1 mixture of ethyl acetate and hexane then ethyl acetate alone and then finally using a 20:1 mixture of ethyl acetate and methanol respectively. The material thus obtained was triturated with diethyl ether and then dried to give N-{[2-chloro-3-(trifluoromethyl)phenyl]methyl}-3-methyl-2-oxo-1,3-oxazolidine-4-carboxamide (0.280 g). LC/MS [M+H]⁺=337/339, retention time=2.37 minutes.

Enantiomeric excess=99.9%, as determined by chiral chromatography method E, indicative of (4S)-N-{[2-chloro-3-(trifluoromethyl)phenyl]methyl}-3-methyl-2-oxo-1,3-oxazolidine-4-carboxamide

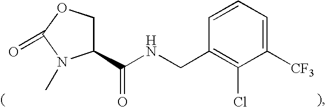

retention time=7.61 minutes.

The N¹-{[2-chloro-3-(trifluoromethyl)phenyl]methyl}-N²-methylserinamide used in the above procedure was prepared as follows:

(i) N-{[(1,1-Dimethylethyl)oxy]carbonyl}-N-methyl-L-serine (2.2 g, 10 mmol) was dissolved in dichloromethane (60 ml) and treated with N-(3-dimethylaminopropyl)-N′-ethylcarbodiimide hydrochloride (2.3 g, 12 mmol) and 1-hydroxybenzotriazole (1.62 g, 12 mmol). The mixture was stirred at room temperature for 15 minutes and then a solution of {[2-chloro-3-(trifluoromethyl)phenyl]methyl}amine (2.09 g, 10 mmol) in dichloromethane (10 ml) was added and the mixture was stirred for an additional 1 hr at room temperature. The mixture was then washed sequentially with saturated aqueous sodium hydrogen carbonate solution, water, and brine. Drying and evaporation gave crude 1,1-dimethylethyl [2-({[2-chloro-3-(trifluoromethyl)phenyl]methyl}amino)-1-(hydroxymethyl)-2-oxoethyl]methylcarbamate which was used without further purification in the next step.

(ii) Crude 1,1-dimethylethyl [2-({[2-chloro-3-(trifluoromethyl)phenyl]methyl}amino)-1-(hydroxymethyl)-2-oxoethyl]methylcarbamate (4.1 g, ˜10 mmol) was dissolved in dichloromethane (20 ml) and treated with trifluoroacetic acid (10 ml). The mixture was stirred at room temperature under argon for ˜1 hr and then the solvent was evaporated and the residue was partitioned between dichloromethane and saturated aqueous sodium hydrogen carbonate solution (pH 9). The organic layer was separated and washed with brine, dried and then evaporated. The residue was triturated with diethyl ether and dried to give crude N¹-{[2-chloro-3-(trifluoromethyl)phenyl]methyl}-N²-methylserinamide (2.47 g). LC/MS [M+H]⁺=311/313, retention time=0.74 minutes.

### Example 5

### N-{[2-chloro-3-(trifluoromethyl)phenyl]methyl}-5-oxo-4-(phenylmethyl)-3-morpholinecarboxamide (E5) (in a form obtainable or prepared from N-(phenylmethyl)-L-serine)

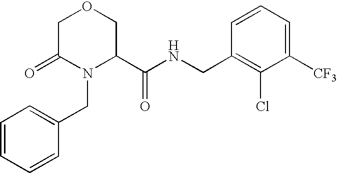

5-Oxo-4-(phenylmethyl)-3-morpholinecarboxylic acid (0.075 g, 0.32 mmol, prepared as described below, starting from N-(phenylmethyl)-L-serine), N-(3-dimethylaminopropyl)-N′-ethylcarbodiimide hydrochloride (0.060 g, 0.32 mmol), 1-hydroxybenzotriazole (0.043 g, 0.32 mmol), {[2-chloro-3-(trifluoromethyl)phenyl]methyl}amine (0.067 g, 0.32 mmol), and N-ethyl morpholine (0.167 ml, 1.28 mmol) were combined in dichloromethane (5 ml) and stirred at room temperature for 3 hours. The mixture was then washed sequentially with saturated aqueous sodium hydrogen carbonate solution and 2N aqueous hydrogen chloride, then the organic layer was separated using a hydrophobic frit and evaporated in vacuo to give N-{[2-chloro-3-(trifluoromethyl)phenyl]methyl}-5-oxo-4-(phenylmethyl)-3-morpholinecarboxamide (0.086 g) as an off-white solid. LC/MS [M+H]⁺=427, retention time=2.83 minutes.

The 5-Oxo-4-(phenylmethyl)-3-morpholinecarboxylic acid used in the above procedure was prepared as follows:

A pre-cooled solution of potassium carbonate (2.1 g, 15.39 mmol) in water (10 ml) was added to a solution of N-(phenylmethyl)-L-serine (1 g, 5.13 mmol) in tetrahydrofuran (10 ml). The mixture was cooled to 0° C. and then chloroacetyl chloride (0.547 ml, 7.18 mmol) was added slowly and stirring continued for 30 minutes. A 50% (wt %) aqueous solution of sodium hydroxide was added (keeping the mixture at 0° C.) until the pH was >13. The mixture was then cooled to −5° C. and then left to stir overnight. Hexane (2×10 ml) was then added to the mixture and the mixture was stirred vigorously for 2 minutes before separating and discarding the organic layers. The aqueous layer was cooled to −10° C. and treated with concentrated aqueous hydrogen chloride until the pH was <2. The resulting slurry was kept at −10° C. and stirred for 2 hrs. The solid was collected by filtration and dried in vacuo (at 45° C.) to give 5-Oxo-4-(phenylmethyl)-3-morpholinecarboxylic acid (0.424 g) as a white solid which was used without further purification. LC/MS [M+H]⁺=235, retention time=1.60 minutes.

### Examples 6-9

In a manner analogous to that described for Example 5 above the compounds tabulated below (Table 1) were prepared by substituting the appropriate amine (or salt thereof) for the {[2-chloro-3-(trifluoromethyl)phenyl]methyl}amine used in the above procedure. All of the amines used to make the compounds shown in Table 1 are available from commercial sources or can be prepared using routes described previously in the chemical literature or analogous methods. The 5-Oxo-4-(phenylmethyl)-3-morpholinecarboxylic acid used in these examples was obtained from commercial methyl 5-oxo-4-(phenylmethyl)-3-morpholinecarboxylate using a standard method for converting a carboxylic ester to the corresponding carboxylic acid (i.e. treatment with aqueous sodium hydroxide in methanol).

TABLE 1

| | | | Re- |
|---|---|---|---|
| Ex- | | | ten- |
| am- | | | tion |
| ple | | [M + | time |
| no. | Chemical name | H]⁺ | (mins) |
| E6 | 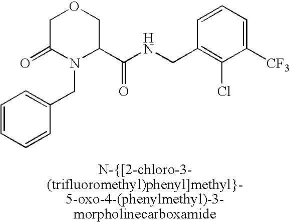 | 427 | 2.80 |
| E7 | 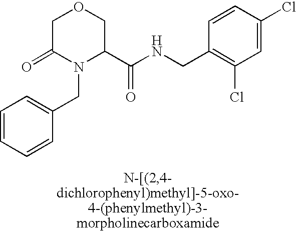 | 393 | 2.76 |
| E8 | 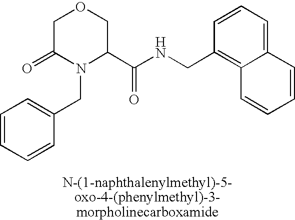 | 375 | 2.66 |
| E9 | 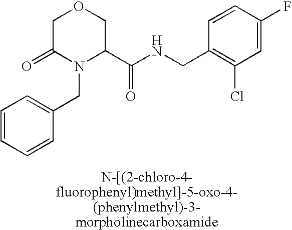 | 377 | 2.57 |

### Example 10

### N-{[2-chloro-3-(trifluoromethyl)phenyl]methyl}-4-methyl-5-oxo-3-morpholinecarboxamide (E10) (in a form obtainable or prepared from N-{[(1,1-dimethylethyl)oxy]carbonyl}-N-methyl-L-serine)

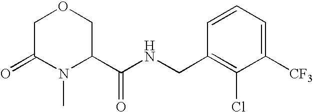

N²-(bromoacetyl)-N¹-{[2-chloro-3-(trifluoromethyl)phenyl]methyl}-N²-methylserinamide (0.660 g, 1.53 mmol, prepared as described below, starting from N-{[(1,1-dimethylethyl)oxy]carbonyl}-N-methyl-L-serine), was dissolved in tetrahydrofuran (25 ml) and treated with potassium carbonate (0.663 g, 4.59 mmol). The mixture was stirred overnight at room temperature. LC/MS indicated that only a small amount of product had formed, and that mostly unreacted starting material remained, and heating the mixture at 50° C. for a further 8 hours made little difference. The solvent was evaporated in vacuo, and the residue taken up in isopropyl alcohol and treated with potassium hydroxide (0.086 g, 1.53 mmol). The mixture was then stirred overnight at room temperature. Evaporation in vacuo gave a residue which was purified by mass-directed automated HPLC to give the product as a pale yellow hydroscopic solid. This was then triturated with diethyl ether to give N-{[2-chloro-3-(trifluoromethyl)phenyl]methyl}-4-methyl-5-oxo-3-morpholinecarboxamide (0.061 g). LC/MS [M+H]⁺=350.93, retention time=2.28 minutes.

The N²-(bromoacetyl)-N¹-{[2-chloro-3-(trifluoromethyl)phenyl]methyl}-N²-methyl-serinamide used in the procedure described above was prepared as follows:

(i) N-{[(1,1-dimethylethyl)oxy]carbonyl}-N-methyl-L-serine (2.5 g, 11.4 mmol), 2-chloro-3-trifluoromethyl benzylamine (2.38 g, 11.4 mmol), and 2-ethoxy-1-ethoxycarbonyl-1,2-dihydroquinoline (2.8 g, 11.63 mmol) were stirred together in dichloromethane (50 ml) at room temperature for 2.5 hrs. The mixture was then washed with 2N aqueous hydrogen chloride (3×50 ml) and then with saturated aqueous sodium hydrogen carbonate solution (50 ml). Separation of the organic phase using a hydrophobic frit and evaporation in vacuo gave 1,1-dimethylethyl [2-({[2-chloro-3-(trifluoromethyl)phenyl]methyl}amino)-1-(hydroxymethyl)-2-oxoethyl]methylcarbamate as an off-white solid (3.5 g) which was used in the next step without further purification.

LC/MS [M+H]⁺=311.00, retention time=2.74 minutes.

(ii) 1,1-Dimethylethyl [2-({[2-chloro-3-(trifluoromethyl)phenyl]methyl}amino)-1-(hydroxymethyl)-2-oxoethyl]methylcarbamate (3.5 g, 8.5 mmol) was dissolved in dichloromethane (10 ml) and treated with a 1M solution of hydrogen chloride in diethyl ether. The mixture was stirred at room temperature for 7 hrs, then treated with a further aliquot (10 ml) of the 1M ethereal hydrogen chloride solution and stirred overnight at room temperature. Evaporation and trituration of the residue with a 1:1 mixture of hexane and ethyl acetate gave N¹-{[2-chloro-3-(trifluoromethyl)phenyl]methyl}-N²-methylserinamide hydrochloride (2.5 g) as a white solid after drying.

LC/MS [M+H]⁺=310.98, retention time=1.52 minutes.

(iii) N¹-{[2-chloro-3-(trifluoromethyl)phenyl]methyl}-N²-methylserinamide hydrochloride (1 g, 2.89 mmol) and triethylamine (0.79 ml, 5.78 mmol) were suspended in tetrahydrofuran (20 ml) and treated with bromo acetyl bromide (0.247 ml, 2.89 mmol). The mixture was stirred at room temperature for 4.5 hrs and then reduced in vacuo and the residue was partitioned between dichloromethane (25 ml) and water (40 ml). The organic phase was separated using a hydrophobic frit and then washed sequentially with 2N aqueous hydrogen chloride (30 ml) and saturated aqueous sodium hydrogen carbonate. Evaporation of the solvent and purification by automated flash-silica gel column chromatography, eluting with a 0-10% gradient of methanol in dichloromethane, gave N²-(bromoacetyl)-N¹-{[2-chloro-3-(trifluoromethyl)phenyl]methyl}-N²-methyl-serinamide (0.666 g) as a clear gum.

### Mass-Directed Automated HPLC

Where indicated in the above examples, purification by mass-directed automated HPLC was carried out using the following apparatus and conditions:

### Hardware

Waters 2525 Binary Gradient Module

Waters 515 Makeup Pump

Waters Pump Control Module

Waters 2767 Inject Collect

Waters Column Fluidics Manager

Waters 2996 Photodiode Array Detector

Waters ZQ Mass Spectrometer

Gilson 202 fraction collector

Gilson Aspec waste collector

### Software

Waters MassLynx version 4 SP2

### Column

The columns used are Waters Atlantis, the dimensions of which are 19 mm×100 mm (small scale) and 30 mm×100 mm (large scale). The stationary phase particle size is 5 μm.

### Solvents

A: Aqueous solvent=Water+0.1% Formic Acid

B: Organic solvent=Acetonitrile+0.1% Formic Acid

Make up solvent=Methanol:Water 80:20

Needle rinse solvent=Methanol

### Methods

There are five methods used depending on the analytical retention time of the compound of interest. They have a 13.5-minute runtime, which comprises a 10-minute gradient followed by a 3.5 minute column flush and re-equilibration step.

Large/Small Scale 1.0-1.5=5-30% B

Large/Small Scale 1.5-2.2=15-55% B

Large/Small Scale 2.2-2.9=30-85% B

Large/Small Scale 2.9-3.6=50-99% B

Large/Small Scale 3.6-5.0=80-99% B (in 6 minutes followed by 7.5 minutes flush and re-equilibration)

### Flow Rate

All of the above methods have a flow rate of either 20 mls/min (Small Scale) or 40 mls/min (Large Scale).

### Chiral HPLC

Apparatus and conditions used to characterize enantiomeric purity of selected samples was as follows:

### Method (A)

Instrument: Agilent 1100 Series Liquid Chromatogram

Column: Chiralpak AD (250 mm×4.6 mm; 10 um particle size)

Mobile phase: Heptane:absolute ethanol (70:30) v/v pump-mixed

Flow rate: 1 ml/min

Temperature: Ambient

U.V. Wavelength: 215 nm

### Method (B)

Instrument: Agilent 1100 Series Liquid Chromatogram

Column: Chiralpak AD (250 mm×4.6 mm; 10 um particle size)

Mobile phase: Heptane:absolute ethanol (50:50) v/v pump-mixed

Flow rate: 1 ml/min

Temperature: Ambient

U.V. Wavelength: 215 nm

### Method (C)

Instrument: Agilent 1100 Series Liquid Chromatogram

Column: Chiralpak AD (250 mm×4.6 mm; 10 um particle size)

Mobile phase: Heptane:absolute ethanol (80:20) v/v pump-mixed

Flow rate: 1 ml/min

Temperature: Ambient

U.V. Wavelength: 215 nm

### Method (D)

Instrument: Agilent 1100 Series Liquid Chromatogram

Column: Chiralpak AS (250 mm×4.6 mm; 10 um particle size)

Mobile phase: Heptane:absolute ethanol (80:20) v/v pump-mixed

Flow rate: 1 ml/min

Temperature: Ambient

U.V. Wavelength: 215 nm

### Method (E)

Instrument: Berger SFC Analytical

Column: Chiralcel OD (250 mm×4.6 mm; 10 um particle size)

Mobile phase: Carbon dioxide and methanol (SFC iso 90:10)

Flow rate: 2.35 ml/min

Temperature/pressure: 38° C./100 Bar

U.V. Wavelength: 215 nm

### Liquid Chromatography/Mass Spectrometry

Analysis of the above Examples by Liquid Chromatography/Mass Spectrometry (LC/MS) was carried out using the following apparatus and conditions:

### Hardware

Agilent 1100 Gradient Pump

Agilent 1100 Autosampler

Agilent 1100 DAD Detector

Agilent 1100 Degasser

Agilent 1100 Oven

Agilent 1100 Controller

Waters ZQ Mass Spectrometer

Sedere Sedex 85

### Software

Waters MassLynx version 4.0 SP2

### Column

The column used is a Waters Atlantis, the dimensions of which are 4.6 mm×50 mm. The stationary phase particle size is 3 μm.

### Solvents

A: Aqueous solvent=Water+0.05% Formic Acid

B: Organic solvent=Acetonitrile+0.05% Formic Acid

### Method

The generic method used has a 5 minute runtime.

| Time/min | % B |
|---|---|
| 0 | 3 |
| 0.1 | 3 |
| 4 | 97 |
| 4.8 | 97 |
| 4.9 | 3 |
| 5.0 | 3 |

The above method has a flow rate of 3 ml/mins.

The injection volume for the generic method is 5 ul.

The column temperature is 30 deg.

The UV detection range is from 220 to 330 nm.

### Pharmacological Data

Compounds of the invention may be tested for in vitro biological activity at the P2X7 receptor in accordance with the following studies:

### Ethidium Accumulation Assay

Studies were performed using NaCl assay buffer of the following composition (in mM): 140 mM NaCl, HEPES 10, N-methyl-D-glucamine 5, KCl 5.6, D-glucose 10, CaCl₂ 0.5 (pH 7.4). HEK293 cells, expressing human recombinant P2X7 receptors, were grown in poly-L-lysine pretreated 96 well plates for 18-24 h. (The cloning of the human P2X7 receptor is described in U.S. Pat. No. 6,133,434). The cells were washed twice with 350 μl of assay buffer before addition of 50 μl of antagonist. The cells were then incubated at room temperature (19-21° C.) for 30 min before addition of ATP and ethidium (100 μM final assay concentration). The ATP concentration was chosen to be close to the EC₈₀ for the receptor type and was 1 mM for studies on the human P2X7 receptor. Incubations were continued for 8 or 16 min and were terminated by addition of 25 μl of 1.3M sucrose containing 5 mM of the P2X7 receptor antagonist reactive black 5 (Aldrich). Cellular accumulation of ethidium was determined by measuring fluorescence (excitation wavelength of 530 nm and emission wavelength of 620 nm) from below the plate with a Canberra Packard Fluorocount (Pangbourne, UK). Antagonist pIC₅₀ values for blocking ATP responses were determined using iterative curve fitting techniques.

### Fluorescent Imaging Plate Reader (FLIPR) Ca Assay

Studies were performed using NaCl assay buffer of the following composition (in mM) for human P2X7: 137 NaCl; 20 HEPES; 5.37 KCl; 4.17 NaHCO₃; 1 CaCl₂; 0.5 MgSO₄; and 1 g/L of D-glucose (pH 7.4).

HEK293 cells, expressing human recombinant P2X7 receptors, were grown in poly-L-lysine pretreated 384 well plates for 42-48 h. (The cloning of the human P2X7 receptor is described in U.S. Pat. No. 6,133,434). The cells were washed three times with 80 μl of assay buffer, loaded for 1 h at 37° C. with 2 μM Fluo4 (Teflabs), washed three times again, and left with 30 μl buffer before the addition of 10 μl of 4× concentrated antagonist. The cells were then incubated at room temperature for 30 mins before addition (online, by FLIPR384 or FLIPR3 instrument (Molecular Devices)) of Benzoylbenzoyl-ATP (BzATP) 60 μM final assay concentration. The BzATP concentration was chosen to be close to the EC₈₀ for the receptor type. Incubations and reading were continued for 90 sec, and intracellular calcium increase was determined by measuring fluorescence (excitation wavelength of 488 nm and emission wavelength of 516 nm) from below the plate, with FLIPR CCD camera. Antagonist pIC₅₀ values for blocking BzATP responses were determined using iterative curve fitting techniques.

The compounds of Examples 1-10 were tested in the FLIPR Ca Assay and/or the Ethidium Accumulation Assay for human P2X7 receptor antagonist activity and found to have pIC50 values >4.7 in the FLIPR Ca Assay and/or pIC50 values >5.5 in the Ethidium Accumulation Assay. Examples E1, E3, E4 and E10 were found to have pIC50 values of about 7.0 or more in the Ethidium Accumulation Assay.

1-18. (canceled)

19. A compound of formula (I): 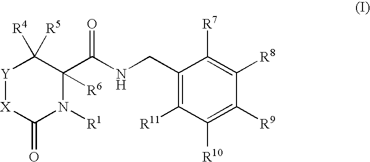
wherein:
R¹ represents C₁₋₆ alkyl, C₂₋₆ alkenyl, C₂₋₆ alkynyl, C₃₋₆ cycloalkyl, C₃₋₆ cycloalkylmethyl-, pyridinylmethyl- or benzyl, any of which is optionally substituted with 1, 2 or 3 halogen atoms; or an unsubstituted phenyl;
X represents O or —(CR²R³)—;
Y represents a bond or O;
such that when X is O, Y represents a bond; and when X is —(CR²R³)—, Y represents O;
R² and R³ independently represent hydrogen, C₁₋₆ alkyl, C₆₋₁₀ arylmethyl- or C₃₋₆ cycloalkylmethyl-; and any of said C₁₋₆ alkyl, C₆₋₁₀ arylmethyl- or C₃₋₆ cycloalkylmethyl- is optionally substituted with 1, 2 or 3 halogen atoms;
R⁴, R⁵ and R⁶ independently represent hydrogen, fluorine or methyl; and
R⁷, R⁸, R⁹, R¹⁰ and R¹¹ independently represent hydrogen, halogen, cyano, C₁₋₆ alkyl, C₂₋₆ alkenyl, C₂₋₆ alkynyl, C₃₋₆ cycloalkyl or phenyl, and any of said C₁₋₆ alkyl, C₂₋₆ alkenyl, C₂₋₆ alkynyl, C₃₋₆ cycloalkyl or phenyl is optionally substituted with 1, 2 or 3 halogen atoms;
or R¹⁰ and R¹¹ together with the carbon atoms to which they are attached form a benzene ring which is optionally substituted with 1, 2 or 3 halogen atoms;
with the proviso that when R⁷ and R¹¹ are both selected from hydrogen or fluorine, at least one of R⁸, R⁹ and R¹⁰ is a halogen atom.

20. The compound according to claim 19, wherein R¹ represents unsubstituted methyl, ethyl or benzyl.

21. The compound according to claim 20, wherein R¹ represents methyl or ethyl.

22. The compound according to claim 19, wherein R² and R³ both represent hydrogen.

23. The compound according to claim 19, wherein R⁴, R⁵ and R⁶ all represent hydrogen.

24. The compound according to claim 19, wherein R⁷, R⁸, R⁹, R¹⁰ and R¹¹ independently represent hydrogen, halogen, methyl or trifluoromethyl; or R¹⁰ and R¹¹ together with the carbon atoms to which they are attached form an unsubstituted benzene ring.

25. The compound according to claim 19, wherein R⁷, R⁸, R⁹, R¹⁰ and R¹¹ independently represent hydrogen, chlorine, fluorine, bromine, methyl or trifluoromethyl.

26. The compound according to claim 19, wherein:
R¹ represents unsubstituted methyl, ethyl or benzyl;
X represents O or —CH₂—;
Y represents a bond or O;
such that when X is O, Y represents a bond; and when X is —CH₂—, Y represents O;
R⁴, R⁵ and R⁶ all represent hydrogen; and
R⁷, R⁸, R⁹, R¹⁰ and R¹¹ independently represent hydrogen, chlorine, fluorine, bromine, methyl or trifluoromethyl; or R¹⁰ and R¹¹ together with the carbon atoms to which they are attached form an unsubstituted benzene ring.

27. The compound according to claim 26, wherein:
R⁷, R⁸ and R⁹ are hydrogen, R¹⁰ is trifluoromethyl, and R¹¹ is chlorine, or
R⁷, R⁸ and R¹⁰ are hydrogen, and R⁹ and R¹¹ are chlorine, or
R⁷, R⁸ and R¹⁰ are hydrogen, R⁹ is fluorine, and R¹¹ is chlorine.

28. A compound which is:
(4R)-N-[(2-chloro-4-fluorophenyl)methyl]-3-ethyl-2-oxo-1,3-oxazolidine-4-carboxamide;
N-{[2-chloro-3-(trifluoromethyl)phenyl]methyl}-3-methyl-2-oxo-1,3-oxazolidine-4-carboxamide;
(4R)-N-{[2-chloro-3-(trifluoromethyl)phenyl]methyl}-3-methyl-2-oxo-1,3-oxazolidine-4-carboxamide;
N-{[2-chloro-3-(trifluoromethyl)phenyl]methyl}-5-oxo-4-(phenylmethyl)-3-morpholinecarboxamide;
N-{[2-chloro-3-(trifluoromethyl)phenyl]methyl}-5-oxo-4-(phenylmethyl)-3-morpholinecarboxamide;
N-[(2,4-dichlorophenyl)methyl]-5-oxo-4-(phenylmethyl)-3-morpholinecarboxamide;
N-(1-naphthalenylmethyl)-5-oxo-4-(phenylmethyl)-3-morpholinecarboxamide; or
N-[(2-chloro-4-fluorophenyl)methyl]-5-oxo-4-(phenylmethyl)-3-morpholinecarboxamide.

29. A compound which is:
(4S)-N-[(2-chloro-4-fluorophenyl)methyl]-3-ethyl-2-oxo-1,3-oxazolidine-4-carboxamide;
(4S)-N-{[2-chloro-3-(trifluoromethyl)phenyl]methyl}-3-methyl-2-oxo-1,3-oxazolidine-4-carboxamide; or
N-{[2-chloro-3-(trifluoromethyl)phenyl]methyl}-4-methyl-5-oxo-3-morpholinecarboxamide.

30. A compound of formula (IB): 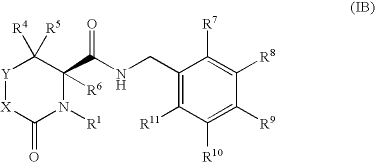
wherein:
R¹ represents methyl or ethyl;
X represents O or —(CR²R³)—;
Y represents a bond or O;
such that when X is O, Y represents a bond; and when X is —(CR²R³)—, Y represents O;
R², R³, R⁴, R⁵ and R⁶ all represent hydrogen; and
R⁷, R⁸, R⁹, R¹⁰ and R¹¹ independently represent hydrogen, halogen, cyano, C₁₋₆ alkyl, C₂₋₆ alkenyl, C₂₋₆ alkynyl, C₃₋₆ cycloalkyl or phenyl, and any of said C₁₋₆ alkyl, C₂₋₆ alkenyl, C₂₋₆ alkynyl, C₃₋₆ cycloalkyl or phenyl is optionally substituted with 1, 2 or 3 halogen atoms; or R¹⁰ and R¹¹ together with the carbon atoms to which they are attached form a benzene ring which is optionally substituted with 1, 2 or 3 halogen atoms;
with the proviso that when R⁷ and R¹¹ are both selected from hydrogen or fluorine, at least one of R⁸, R⁹ and R¹⁰ is a halogen atom;
and wherein more than 50% by molarity of the compound of formula (IB) has the indicated stereochemistry at the ring-carbon atom bonded to R⁶.

31. The compound according to claim 30, wherein:
R⁷, R⁸ and R⁹ are hydrogen, R¹⁰ is trifluoromethyl, and R¹¹ is chlorine, or
R⁷, R⁸ and R¹⁰ are hydrogen, and R⁹ and R¹¹ are chlorine, or
R⁷, R⁸ and R¹⁰ are hydrogen, R⁹ is fluorine, and R¹¹ is chlorine.

32. A pharmaceutical composition which comprises the compound of formula (I), as defined in claim 19, and a pharmaceutically acceptable carrier or excipient.

33. A method of treating a human suffering from pain, inflammation or a neurodegenerative disease, which method comprises administering to said human an effective amount of the compound of formula (I) as defined in claim 19.

34. A method of treating a human suffering from inflammatory pain, neuropathic pain or visceral pain, which method comprises administering to said human an effective amount of the compound of formula (I) as defined in claim 19.
